(19) United States
(12) Patent Application Publication
(10) Pub. No.: US 2017/0335272 A1
(43) Pub. Date: Nov. 23, 2017

(54) CLOSE-SYSTEM CELL ISOLATION METHOD, CLOSE-SYSTEM CELL CULTURE BAG, AND CLOSE-SYSTEM CELL ISOLATION DEVICE

(71) Applicant: NAVI BIO-THERAPEUTICS. INC., Kaohsiung City (TW)

(72) Inventors: Bor-Yu Tsai, Kaohsiung City (TW); Long-Sun Huang, Kaohsiung City (TW); Bor-Leun Chiang, Kaohsiung City (TW)

(21) Appl. No.: 15/248,484

(22) Filed: Aug. 26, 2016

(30) Foreign Application Priority Data
May 18, 2016 (CN) 201610331420.7

(51) Int. Cl. C12M 1/00; C12M 3/00; C12N 13/00

(52) U.S. Cl. CPC C12M 47/04; C12N 13/00; C12M 21/08; C12M 23/14; C12M 23/58

(57) ABSTRACT

A close-system cell isolation device includes a first culture bag, a second culture bag, immunomagnetic beads, and a connecting tube. The first culture bag and the second culture bag are closely connected with each other through the connecting tube. The first culture bag has the immunomagnetic beads and cells. When the first culture bag is forced by a magnetic force and an external force, the cells uncaptured by the immunomagnetic beads are moved from the first culture bag to the second culture bag through the connecting tube by the external force, and the cells captured by the immunomagnetic beads are retained in the first culture bag by the magnetic force. Accordingly, the cell isolation can be performed without contamination.

### CROSS-REFERENCES TO RELATED APPLICATIONS

This non-provisional application claims priority under 35 U.S.C. §119(a) on Patent Application No. 201610331420.7 filed in China, P.R.C. on May 18, 2016, the entire contents of which are hereby incorporated by reference.

### BACKGROUND

### Technical Field

The instant disclosure relates to a cell isolation device, in particular, to a close-system cell isolation method, a close-system cell culture bag, and a close-system cell isolation device.

### Related Art

In order to cure diseases, to improve agricultural productivity, or to improve the environments, the advances of biotechnology and its related products go faster and faster. Specifically, cell isolation is an important technique for obtaining purified cells for further usage or experiments.

Common cell isolation methods include differential centrifugation, density gradient centrifugation, flow cytometry, cell electrophoresis, etc. However, to obtain an isolated cell sample with improved purity, these methods have to be repeated for several times. As a result, the conventional method is insufficient and time consuming. Moreover, in the conventional operation, after the cell sample is centrifuged, an operator has to use a pipette to suck up the suspension liquid of the sample in a sterile bench (hood) to perform the cell isolation. However, because such operation is performed in an open space, the cell samples may still be highly possibly contaminated.

### SUMMARY

In one embodiment, a close-system cell isolation device comprises a plurality of culture bags, a plurality of immunomagnetic beads, and a connecting tube. Each of the culture bags comprises a bag body, two normal close openings, and a normal close connecting port. The bag body has a receiving space for culturing a plurality of cells. The two normal close openings are at the bag body and communicating with the receiving space. The two normal close openings are for inputting or outputting a culture liquid of the cells. The normal close connecting port is at the bag body and communicating with the receiving space. The culture bags comprise a first culture bag and at least one second culture bag. The immunomagnetic beads are in the receiving space of the first culture bag. Each of the immunomagnetic beads comprises a plurality of binders for capturing a specific cell (at least one or all of the cells). The connecting tube is closely connected between the normal close connecting port of the first culture bag and the normal close connecting port of the second culture bag. When the first culture bag is forced by a magnetic force and an external force, cells in the first culture bag and uncaptured by the immunomagnetic beads are moved to the second culture bag (one of the at least one second culture bag) through the connecting tube by the external force.

In one embodiment, a close-system cell isolation method comprises: providing a magnetic force to a first culture bag having a plurality of cells and a plurality of immunomagnetic beads, and providing a centrifugation force to the first culture bag by the rotation of a rotating plate, so that cells uncaptured by the immunomagnetic beads are moved from the first culture bag to the second culture bag through the connecting tube. Wherein, each of the immunomagnetic beads comprises a plurality of binders, and the binders capture a plurality of numbers of the cells.

In another embodiment, a close-system cell isolation method comprises: providing a magnetic force to a first culture bag having a plurality of cells and a plurality of immunomagnetic beads, and allowing the first culture bag to be at a first setting position and allowing a second culture bag to be at a second setting position which is lower than the first setting position, so that cells uncaptured by the immunomagnetic beads are moved from the first culture bag to the second culture bag through a connecting tube. Wherein, each of the immunomagnetic beads comprises a plurality of binders, and the binders capture a plurality of numbers of the cells.

In yet another embodiment, a close-system cell isolation method comprises: providing a magnetic force to a first culture bag having a plurality of cells and a plurality of immunomagnetic beads, and inputting a culture liquid into the first culture bag so that cells uncaptured by the immunomagnetic beads are moved from the first culture bag to a second culture bag through a connecting tube. Wherein, each of the immunomagnetic beads comprises a plurality of binders, and the binders capture a plurality of numbers of the cells.

In a further embodiment, a close-system cell culture bag comprises a bag body, two normal close openings, and a normal close connecting port. The bag body has a receiving space for culturing a plurality of cells. The two normal close openings are at the bag body and communicating with the receiving space for inputting or outputting a culture liquid of the cells. The normal close connecting port is at the bag body and communicating with the receiving space.

Wherein, the normal close connecting port is closely connected to one of the ends of a connecting tube. In some embodiments, the connecting tube may be a one-to-one tube; while in some other embodiments, the connecting tube may be a one-to-many tube.

As above, a close culture system can be provided, and the cells inside the system can be isolated and purified by the culture bags closely connected with each other without the contamination and provided for further cell culture procedure.

### BRIEF DESCRIPTION OF THE DRAWINGS

The disclosure will become more fully understood from the detailed description given herein below for illustration only, and thus not limitative of the disclosure, wherein:

FIG. 1 illustrates a perspective view of a first embodiment of a close-system cell isolation device according to the instant disclosure;

FIG. 2 illustrates a perspective view of a second embodiment of a close-system cell isolation device according to the instant disclosure;

FIG. 3 illustrates a perspective view of a third embodiment of a close-system cell isolation device according to the instant disclosure;

FIG. 4 illustrates a perspective view of an embodiment of the connecting tube shown in FIG. 3;

FIGS. 5A and 5B illustrate schematic operational views showing an embodiment of the operation of cell isolation by using the close-system cell isolation device shown in FIG. 1;

FIG. 6 illustrates a flowchart of a first embodiment of a close-system cell isolation method according to the instant disclosure;

FIG. 7 illustrates a perspective view of a fourth embodiment of a close-system cell isolation device according to the instant disclosure;

FIG. 8 illustrates a flowchart of a second embodiment of a close-system cell isolation method according to the instant disclosure;

FIG. 9 illustrates a perspective view of a fifth embodiment of a close-system cell isolation device according to the instant disclosure;

FIG. 10 illustrates a flowchart of a third embodiment of a close-system cell isolation method according to the instant disclosure;

FIG. 11 illustrates a flowchart of a fourth embodiment of a close-system cell isolation method according to the instant disclosure;

FIG. 12 illustrates a perspective view of a sixth embodiment of a close-system cell isolation device according to the instant disclosure;

FIG. 13 illustrates a flowchart of a fifth embodiment of a close-system cell isolation method according to the instant disclosure;

FIG. 14 illustrates a flowchart of a sixth embodiment of a close-system cell isolation method according to the instant disclosure; and

FIG. 15 illustrates a flowchart of a seventh embodiment of a close-system cell isolation method according to the instant disclosure.

### DETAILED DESCRIPTION

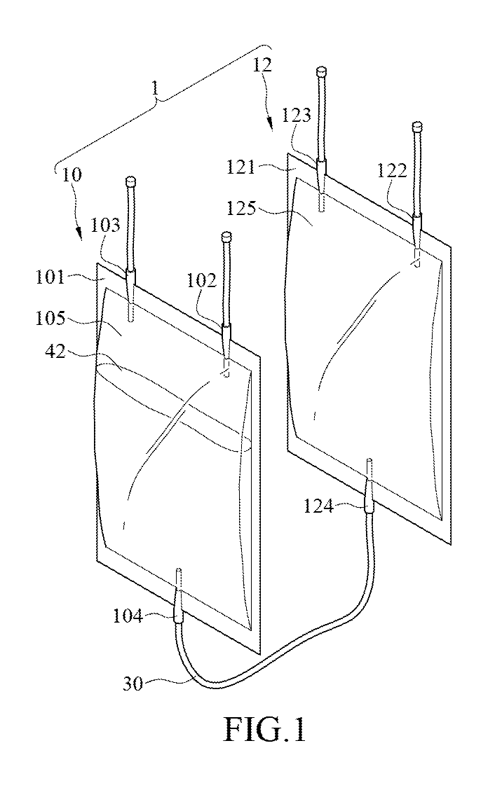

FIG. 1 illustrates a perspective view of a first embodiment of a close-system cell isolation device according to the instant disclosure. In one embodiment, referring to FIG. 1, the cell isolation device 1 is a close system for cell culture and/or cell isolation. The cell isolation device 1 comprises a plurality of culture bags and a connecting tube 30. For the convenience, in the following examples, the number of the culture bags is two, i.e., a first culture bag 10 and a second culture bag 12.

The first culture bag 10 comprises a bag body 101, a first normal close opening 102, a second normal close opening 103, and a normal close connecting port 104. The term “normal close” means the opening (connecting port) is closed in normal conditions, but when being enabled the opening (connecting port) is opened. The bag body 101 has a receiving space 105 for receiving a culture liquid 42 and a plurality of immunomagnetic beads (not shown in FIG. 1). The first normal close opening 102 and the second normal close opening 103 are at the bag body 101, and the first normal close opening 102 and the second normal close opening 103 communicate with the receiving space 105. The first normal close opening 102 and the second normal close opening 103 are for inputting or outputting the culture liquid 42. The normal close connecting port 104 is at the bag body 101 and communicates with the receiving space 105. When the first normal close opening 102, the second normal close opening 103, and the normal close connecting port 104 are all closed, the receiving space 105 becomes a closed (confined) space.

In the cell culture, the culture liquid 42 having a plurality of cells (not shown in FIG. 1) can be inputted in to the receiving space 105 from the first normal close opening 102 and the second normal close opening 103. Specifically, the first normal close opening 102 and the second normal close opening 103 may be provided for feeding the culture liquid 42 or other nutrients based on user's needs during the cell culture, or may be provided for sampling some of the culture liquid 42 to observe and analyze the growth of the cells. The cell culture is performed in a closed system to prevent the cell from being contaminated.

The second culture bag 12 comprises a bag body 121, a first normal close opening 122, a second normal close opening 123, and a normal close connecting port 124. The bag body 121 has a receiving space 125. The first normal close opening 122 and the second normal close opening 123 are at the bag body 121, and the first normal close opening 122 and the second normal close opening 123 communicate with the receiving space 125. The first normal close opening 122 and the second normal close opening 123 are for inputting or outputting the culture liquid 42. The normal close connecting port 124 is at the bag body 121 and communicates with the receiving space 125.

The connecting tube 30 may be closely connected to the normal close connecting port 104 of the first culture bag 10 and the normal close connecting port 124 of the second culture bag 12. Therefore, the receiving space 105 of the first culture bag 10 communicates with the receiving space 125 of the second culture bag 12 through the connecting tube 30.

In the operation of the cell isolation, the connecting tube 30 communicates with the first culture bag 10 and the second culture bag 12 followed by applying a magnetic force and an external force to the first culture bag 10, so that the culture liquid 42 in the receiving space 105 of the first culture bag 10 flows into the receiving space 125 of the second culture bag 12 through the connecting tube 30. In other words, in the operation of the cell isolation, the receiving space 125 of the second culture bag 12 is for receiving all or some of the culture liquid 42 from the first culture bag 10.

In some embodiments, the culture bags may be made of medical grade plastics like polyvinylchloride (PVC), polyethylene (PE), polypropylene (PP), polyethylene terephthalate (PET), polyurethane (PU), ethylene-vinyl acetate copolymer (EVA), fluorinated ethylene propylene (FEP), etc., or may be made of other flexible, sterilizable, and gas permeable materials. Alternatively, the culture bag may be made of a material that can be seen-through, so that a user can observe the condition of the cells in the culture bag.

In some embodiments, the first culture bag 10 and the second culture bag 12 may be identical structures. In other words, the first normal close opening 102 and the second normal close opening 103 are at a first side of the bag body 101 of the first culture bag 10, and the normal close connecting port 104 is at a second side of the bag body 101 of the first culture bag 10, wherein the second side is opposite to the first side. Likewise, the first normal close opening 122 and the second normal close opening 123 are at a first side of the bag body 121 of the second culture bag 12, and the normal close connecting port 124 is at a second side of the bag body 121 of the second culture bag 12, wherein the second side is opposite to the first side.

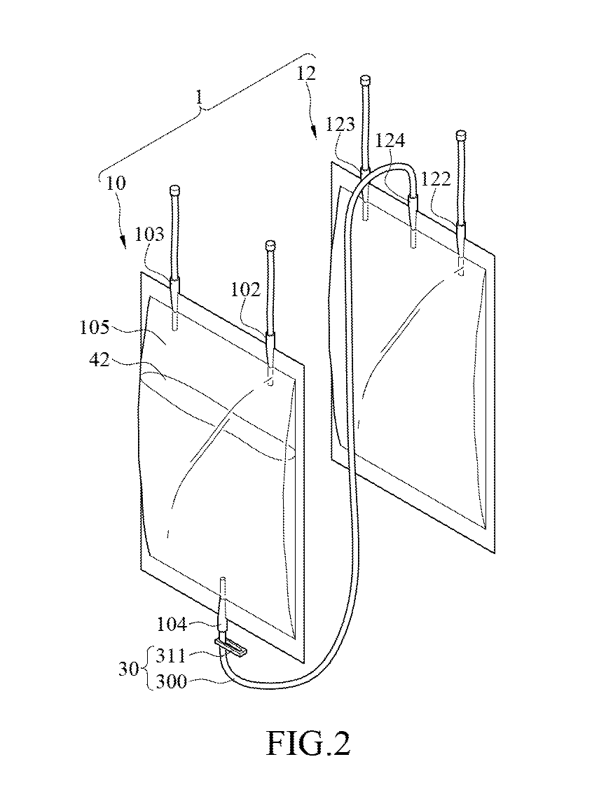

FIG. 2 illustrates a perspective view of a second embodiment of a close-system cell isolation device according to the instant disclosure.

In some embodiments, the structure of the first culture bag may be different from the structure of the second culture bag. Please refer to FIG. 2. The first normal close opening 102 and the second normal close opening 103 are at a first side of the bag body 101 of the first culture bag 10, and the normal close connecting port 104 is at a second side of the bag body 101 of the first culture bag 10, wherein the second side is opposite to the first side; the first normal close opening 122, the second normal close opening 123, and the normal close connecting port 124 are at the same side of the bag body 121 of the second culture bag 12.

In some embodiments, the connecting tube 30 is a closed tube, and each of the ends of the connecting tube 30 has a close adapter.

Please refer to FIGS. 1 and 2. In some embodiments, the connecting tube 30 may be a one-to-one tube, and two ends of the one-to-one tube respectively have close adapters. The connecting tube 30 is for closely connected between the normal close connecting port 104 of the first culture bag 10 and the normal close connecting port 124 of the second culture bag 12, so that the receiving space 105 of the first culture bag 10 is in communication with the receiving space 125 of the second culture bag 12. The connecting tube 30 may be connected to or detached from the culture bag based on the needs.

In some embodiments, the connecting tube 30 comprises a tube 300. One of two ends of the tube 300 is closely connected to the normal close connecting port 104 of the first culture bag 10, and the other end of the tube 300 is closely connected to the normal close connecting port 124 of the second culture bag 12. In some embodiments, the tube 300 may be made of a material which is flexible, sterilizable, and can be seen-through, so that the user can observe the flowing of the culture liquid 42 in the tube 300.

In some embodiments, the connecting tube 30 further comprises a valve 311. The valve 311 is assembled on the tube 300. In this embodiment, the valve 311 is for controlling the flow between the first culture bag 10 and the second culture bag 12 (i.e., the valve 311 can be controlled to allow the first culture bag 10 and the second culture bag 12 to be in communication with each other or not to be in communication with each other). In some embodiments, the valve 311 can be further provided for tuning the amount of the flow in the tube 300.

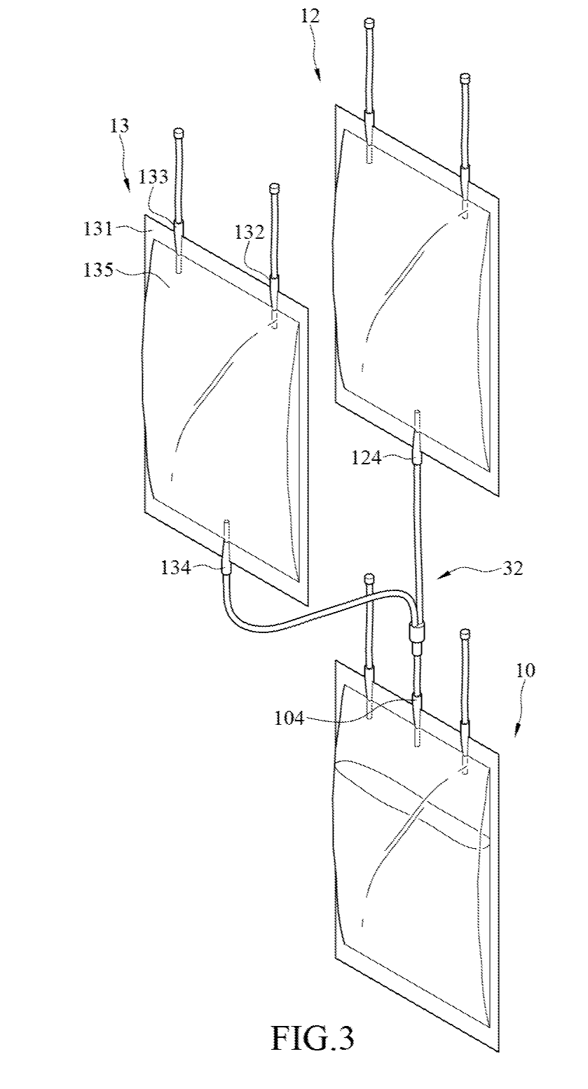

FIG. 3 illustrates a perspective view of a third embodiment of a close-system cell isolation device according to the instant disclosure. In some embodiments, the number of the second culture bag may be plural, meaning that the close-system cell isolation device has three or more culture bags. In the following examples, the number of the culture bags is three, i.e., one first culture bag 10 and two second culture bags 12, 13. In this embodiment, the connecting tube 32 may be a one-to-many tube, and the one-to-many tube is for closely connected the first culture bag 10 between the second culture bags 12, 13. In some embodiments, the second culture bag 12 and the second culture bag 13 may have the identical structure, or may have different structures. In FIG. 3, the first normal close opening 132 and the second normal close opening 133 are at a first side of the bag body 131 of the second culture bag 13, and the normal close connecting port 134 is at a second side of the bag body 131 of the second culture bag 13, wherein the second side is opposite to the first side. The bag body 131 has a receiving space 135.

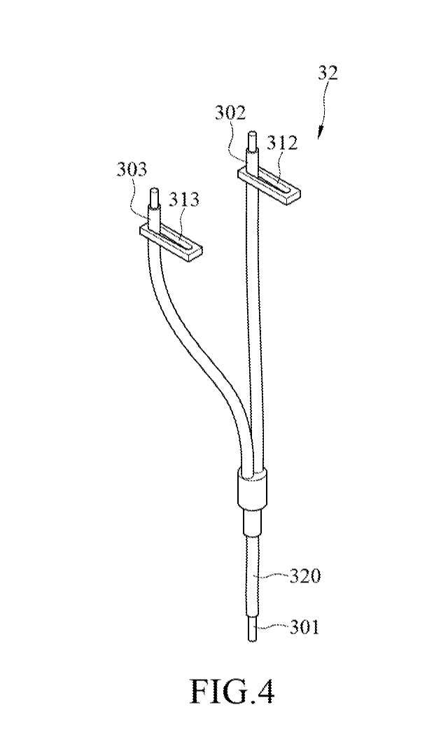

FIG. 4 illustrates a perspective view of an embodiment of the connecting tube shown in FIG. 3. Please refer to FIGS. 3 to 4. The connecting tube 32 is a one-to-two tube, and the one-to-two tube comprises a tube 320, a first end 301, a second end 302, and another second end 303. The first end 301 is closely connected to the normal close connecting port 104 of the first culture bag 10. The second end 302 is closely connected to the normal close connecting port 124 of the second culture bag 12. The second end 303 is closely connected to the normal close connecting port 134 of the second culture bag 13. In some embodiment, the tube 320 may be made of a material which is flexible, sterilizable, and can be seen-through, so that the user can observe the flowing of the culture liquid 42 in the tube 320.

In some embodiments, the connecting tube 32 further comprises valves 312, 313 for controlling the flow of the connecting tube 32. The valve 312 is assembled on a portion of the tube 320 and the portion is close to the second end 302, and the valve 312 is for controlling the first culture bag 10 to be or not to be in communication with the second culture bag 12. The valve 313 is assembled on a portion of the tube 320 and the portion is close to the second end 303, and the valve 313 is for controlling the first culture bag 10 to be or not to be in communication with the second culture bag 13

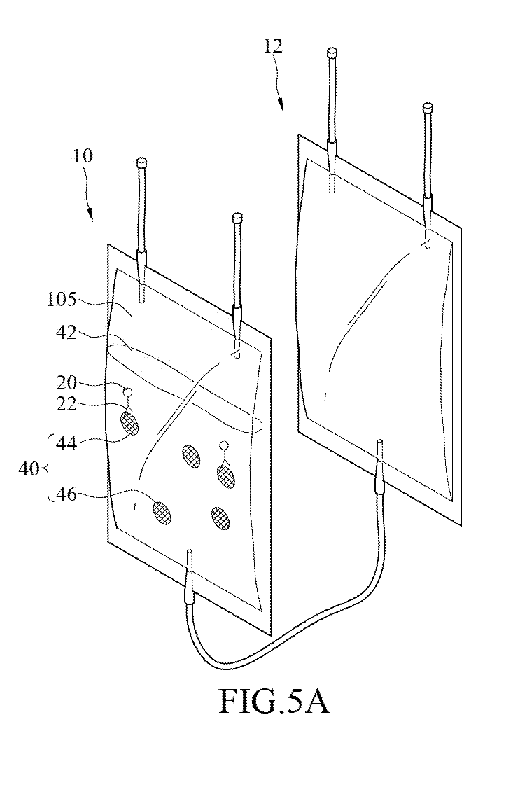

FIGS. 5A and 5B illustrate schematic operational views showing an embodiment of the operation of cell isolation by using the close-system cell isolation device shown in FIG. 1. Please refer to FIG. 5. The immunomagnetic beads 20 are in the receiving space 105 of the first culture bag 10. The immunomagnetic beads 20 are small particles that can be attracted by a magnetic force. In some embodiments, the size of the immunomagnetic bead 20 may be from 50 nm to 4500 nm. In some embodiments, the immunomagnetic beads 20 may be made of a material having strong paramagnetism. Paramagnetism means, when a magnetic field exists, the paramagnetic particles can be accumulated quickly; while when the magnetic field vanishes, the paramagnetic particles can be spread uniformly without accumulation.

The surface of each of the immunomagnetic beads 20 has a plurality of binders 22, and the binders 22 may be combined with the specific surface antigens of specific cells 40 by a specific antibody-antigen combination. In other words, the surface of each of the immunomagnetic beads 20 is coated with different antibodies, so that the immunomagnetic beads 20 can be optionally combined to targets (cells) to be isolated, namely, the surface antigens of the cells can be combined with the specific antibodies coated on the immunomagnetic beads 20. Therefore, the immunomagnetic beads 20 can capture cells having the specific surface antigens. For the sake of clarity, hereinafter, cells that can be captured by the binders 22 of the immunomagnetic beads 20 are called first cells 44, and cells that cannot be captured by the binders 22 of the immunomagnetic beads 20 are called second cells 46.

Please refer to FIG. 5B. when the first culture bag 10 having a plurality of immunomagnetic bead 20 is forced by a magnetic force and an external force, the first cells 44, which have already combined with the immunomagnetic beads 20, will be retained in the receiving space 105 of the first culture bag 10 due to the attraction of the magnetic force, and the second cells 46, which are not combined with the immunomagnetic beads 20 and the culture liquid 42 are moved to the receiving space 125 of the second culture bag 12 through the connecting tube 30 by the external force.

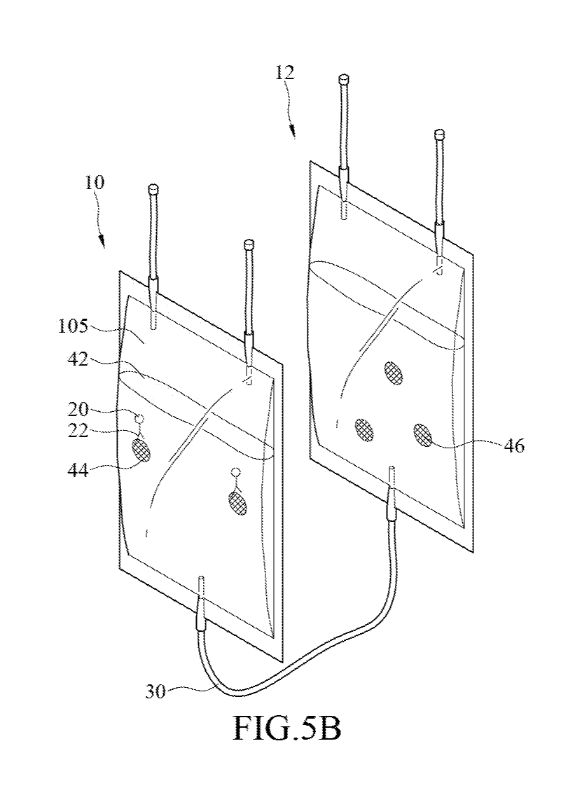

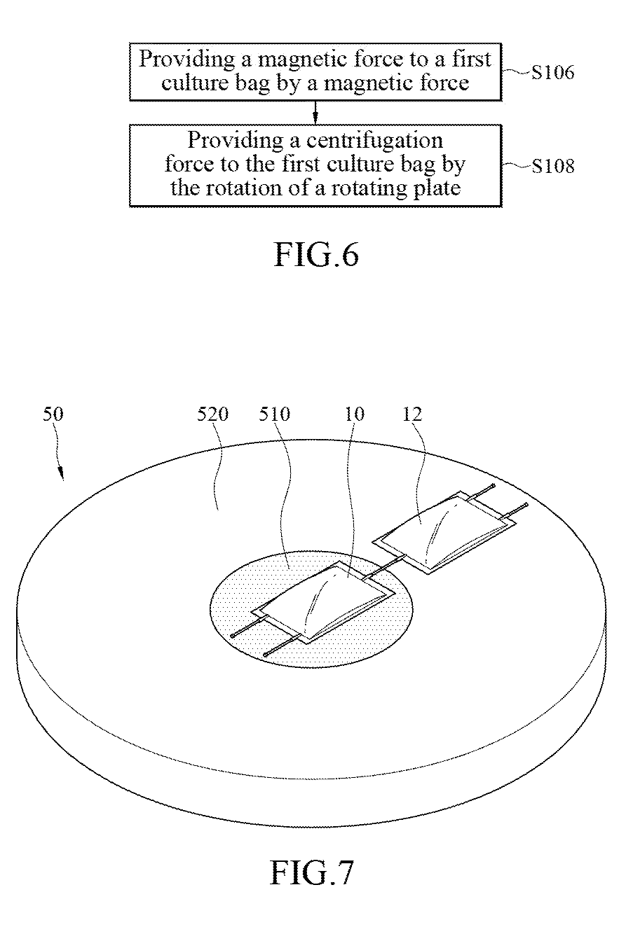

FIG. 6 illustrates a flowchart of a first embodiment of a close-system cell isolation method according to the instant disclosure. In one embodiment, the close-system cell isolation method comprises: providing a magnetic force to the first culture bag 10 by a magnetic device (step S106) and providing a centrifugation force to the first culture bag 10 by the rotation of a rotating plate 50 (step S108). The centrifugation force is one of the embodiments of the external force. The distance between the rotating center of the rotating plate 50 and the position of the rotating plate 50 which the first culture bag 10 is located at is shorter than the distance between the rotating center of the rotating plate 50 and the position of the rotating plate 50 which the second culture bag 12 is located at (namely, the first culture bag 10 is closer to the rotating plate 50 as compared with the second culture bag 12). Therefore, the culture liquid 42 and the second cells 46 flow through the connecting tube 30 by the centrifugation force and further flow into the second culture bag 12; while the first cells 44 are retained in the first culture bag 10 due to the attraction of the magnetic force.

In some embodiments, the magnetic device may be implemented by a permanent magnetic or an electric magnet.

FIG. 7 illustrates a perspective view of a fourth embodiment of a close-system cell isolation device according to the instant disclosure. Please refer to FIGS. 5A, 5B, 6, and 7. In one embodiment, the close-system cell isolation device 1 further comprises the rotating plate 50. The central portion of the rotating plate 50 is a magnetic attraction area 510, and the periphery of the rotating plate 50 is a non-magnetic attraction area 520. In some embodiments, the magnetic device may be implemented by the magnetic attraction area 510. For example, the central portion of the rotating plate 50 may be made from magnet. In some embodiments, the magnetic device may be disposed above or below the central portion of the rotating plate 50, and the magnetic device applies a magnetic force to the central portion of the rotating plate 50 to allow the central portion of the rotating plate 50 to be the magnetic attraction area 510. When the cell isolation is performed, the second culture bag 12 is placed at the non-magnetic attraction area 520, and the first culture bag 10 is placed at the magnetic attraction area 510. The immunomagnetic beads 20 in the receiving space 105 of the first culture bag 10 are attracted by the magnetic force of the magnetic attraction area 510 (step S106). Therefore, when the rotating plate 50 is rotating to apply the centrifugation force to the first culture bag 10 (step S108), the first cells 44 are attracted by the magnetic force to be retained in the receiving space 105 of the first culture bag 10, and the second cells 46 and the culture liquid 42 are forced by the centrifugation force to move to the receiving space 125 of the second culture bag 12 through the connecting tube 30.

In some embodiments, the size of the magnetic attraction area 510 may correspond to the size of the receiving space 105 of the first culture bag 10, so that the immunomagnetic beads 20 in the receiving space 105 can be attracted by the magnetic force. Consequently, the cell purification or isolation can be performed in a better way.

In some embodiments, the immunomagnetic beads 20 may be already in the receiving space 105 of the first culture bag 10 as the first culture bag 10 is purchased. In some embodiments, the immunomagnetic beads 20 may be inputted into the first culture bag 10 to be cultured with the cells 40 during the cell culture procedure. In some embodiments, the immunomagnetic beads 20 are inputted into the first culture bag 10 upon the cell isolation procedure.

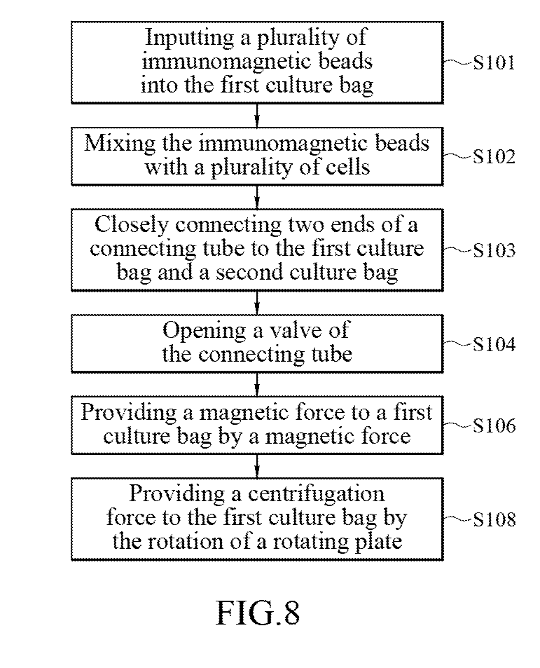

FIG. 8 illustrates a flowchart of a second embodiment of a close-system cell isolation method according to the instant disclosure. In one embodiment, prior to the step S106, the immunomagnetic beads 20 may be inputted into the first culture bag 10 from at least one of the first normal close opening 102 and the second normal close opening 103 (step S101). Next, the immunomagnetic beads 20 are mixed with the cells 40 to allow the binders 22 capturing a plurality of first cells 44 from the cells 40 (step S102). After the reaction (i.e., the binders 22 are completely combined with the first cells 44), the step S106 and steps after the step S106 are performed. In some embodiments, in the step S102, the first culture bag 10 can be shaken thoroughly or stirred by different ways, so that the binders 22 can capture the first cells 44 efficiently.

In some embodiments, the culture bags and the connecting tube 30 are already connected with each other as being purchased.

In some embodiments, the culture bags and the connecting tube 30 are sterilely packed, respectively, as being purchased. The connecting tube 30 is connected to the normal close connecting ports 104, 124 of the culture bags in the cell isolation procedure.

Please refer to FIG. 8. Prior to the step S106, the close adapters at two ends of the connecting tube 30 (the tube 300) may be closely connected to the normal close connecting port 104 of the first culture bag 10 and the normal close connecting port 124 of the second culture bag 12, respectively (step S103).

In some embodiments in which case the connecting tube 30 comprises the valve 311, after the step S103, the valve 311 of the connecting tube 30 may be opened (step S104). Therefore, the tube 300 can be provided as a passage communicating between the first culture bag 10 and the second culture bag 20.

Accordingly, in the cell culture procedure, it is not necessary to connect the connecting tube 30 with the first culture bag 10 and the second culture bag 12; the connecting tube 30 may be connected to or in communication with the culture bags in different stages of the cell isolation procedure, based on needs or preferences.

It is realized that, the order of the steps described above is not a limitation of the instant disclosure, if it is rational, the order of some of the steps may be exchanged, or some of the steps may be performed at the same time. For example, the steps S101 and S102 may be performed before or after the steps S103 and S104, or the steps S101 and S103 may be performed at the same time.

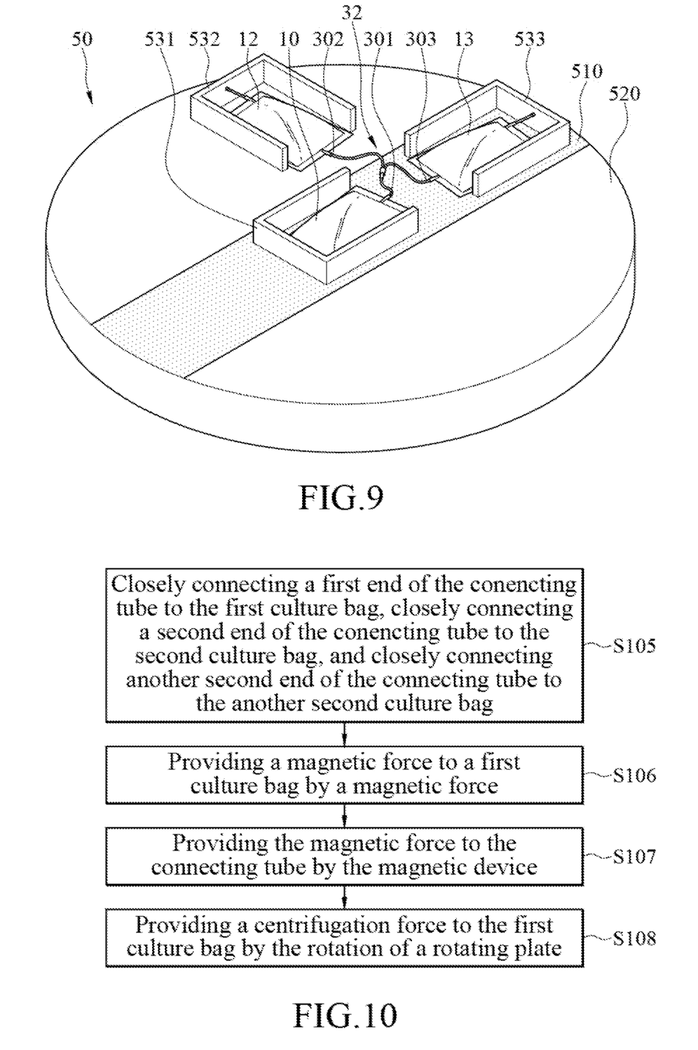

FIG. 9 illustrates a perspective view of a fifth embodiment of a close-system cell isolation device according to the instant disclosure. Please refer to FIGS. 3 and 9. In some embodiments, the rotating plate 50 of the cell isolation device 1 further comprises a plurality of holders 531, 532, 533 for holding the culture bags on the rotating plate 50 and preventing the culture bags from being shifted when the rotating plate 50 is rotating. The holders 531, 532, 533 are disposed on the upper surface of the rotating plate 50. Specifically, the holder 532 is located at the non-magnetic attraction area 520; the holders 531, 533 are located at the magnetic attraction area 510 in which the holder 531 is closer to the central portion of the rotating plate 50. During the cell isolation procedure, the second culture bag 12 is held in the holder 532, so that the second culture bag 12 is not affected by the magnetic force; the first culture bag 10 is held in the holder 531 and in the magnetic attraction area 510; while the second culture bag 13 is held in the holder 533 and in the magnetic attraction area 510. The first culture bag 10 and the two second culture bags 12, 13 are in communication with each other through a one-to-two connecting tube 32. The first end 301 and the second end 303 of the one-to-two connecting tube 32 are near to the magnetic attraction area 510 and forced by the magnetic force, while the second end 302 of the one-to-two connecting tube 32 is not forced by the magnetic force. Therefore, when the rotating plate 50 is rotating to apply the centrifugation force to the first culture bag 10, the first cells 44, which have already combined with the immunomagnetic beads 20, are attracted by the magnetic force so as to be retained in the first culture bag 10 (when the centrifugation force is not greater than the magnetic force), or the first cells 44 are moved to the second culture bag 13 through the second end 303 of the one-to-two tube 32 (when the centrifugation force is greater than the magnetic force); while the second cells 46 and the culture liquid 42 are forced by the centrifugation force so as to be moved to the receiving space 125 of the second culture bag 12 through the connecting tube 32.

FIG. 10 illustrates a flowchart of a third embodiment of a close-system cell isolation method according to the instant disclosure. In some embodiments, prior to the step S106, the first end 301 of the connecting tube 32 is closely connected to the first culture bag 10, the second end 302 of the connecting tube 32 is closely connected to the second culture bag 12, and the second end 303 of the connecting tube 32 is closely connected to the second culture bag 13 (step S105).

In some embodiments, prior to the step S108, the magnetic device (i.e., the magnetic attraction area 510) further provides the magnetic force to the connecting tube 32 (step S107). The order of the steps S106 and step S107 are not limited; the step S106 may be performed before or after the step S107, or the steps S106 and S107 may be performed at the same time.

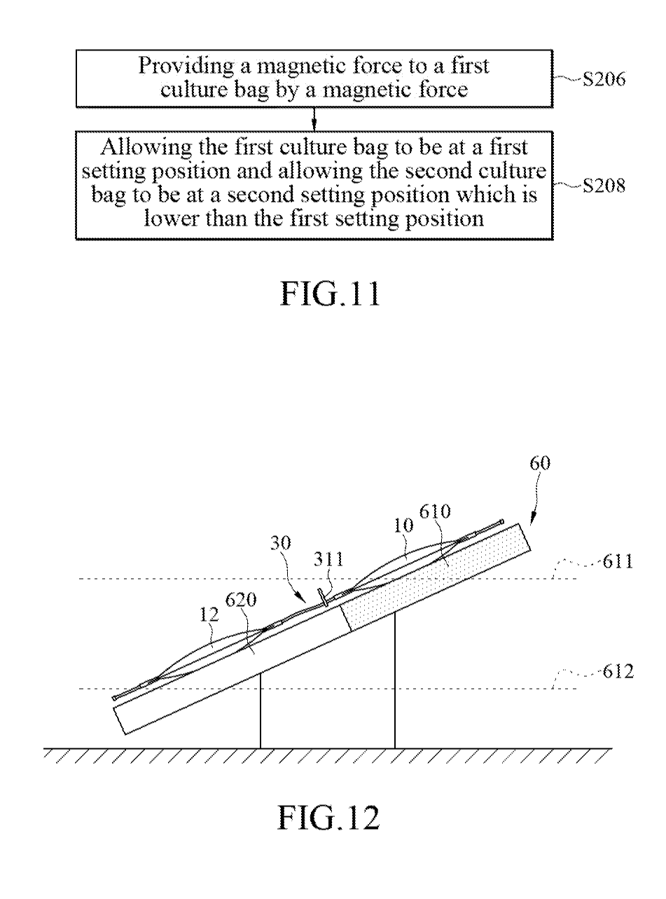

FIG. 11 illustrates a flowchart of a fourth embodiment of a close-system cell isolation method according to the instant disclosure. FIG. 12 illustrates a perspective view of a sixth embodiment of a close-system cell isolation device according to the instant disclosure. In some embodiments, please refer to FIGS. 11 and 12, the close-system cell isolation method comprises providing the magnetic force to the first culture bag 10 by the magnetic device (step S206) and allowing the first culture bag 10 to be at a first setting position 611 and allowing the second culture bag 12 to be at a second setting position 612 which is lower than the first setting position 611 (step S208). The difference of gravitational potential energies between the first setting position 611 and the second setting position 612 is one of the embodiments of the external force. Accordingly, the culture liquid 42 and the second cells 46 in the first culture bag 10 flow through the connecting tube 30 and further flow into the second culture bag 12 due to the gravity, and the first cells 44 are attracted by the magnetic force and retained in the first culture bag 10.

Please refer to FIGS. 5A, 5B, 11, and 12. In some embodiments, the cell isolation device 1 comprises a flat plate 60, a part of the flat plate 60 is the magnetic attraction area 610, and the rest part of the flat plate 60 is the non-magnetic attraction area 620. In some embodiments, the magnetic device may be implemented by the magnetic attraction area 610. For example, the right portion of the flat plate 60 may be made from magnetic. In some embodiment, the magnet device may be in the interior of the right portion of the flat plate 60, and the magnet device applies a magnetic force to the surface of the right portion of the flat plate 60 to allow the right portion of the flat plate 60 to form the magnetic attraction area 610. In the cell isolation procedure, the second culture bag 12 is placed on the non-magnetic attraction area 620, and the first culture bag 10 is placed on the magnetic attraction area 610, so that the immunomagnetic beads 20 in the receiving space 105 can be attracted by the magnetic force provided by the magnetic attraction area 510. Next, the flat plate 60 is tilted to position the first culture bag 10 at the first setting position 611 and to position the second culture bag 12 at the second setting position 612. Therefore, the first culture bag 10 is forced by the magnetic force and the gravity at the same time. In some embodiments, in addition to the flat plate 60, hanging or other ways may be utilized to position the first culture bag 10 at the first setting position 611 which is higher than the second setting position 612 the second culture bag 12 is.

In some embodiments, unlike the single plate structure of the flat plate 60 shown in FIG. 12, the magnetic attraction area 610 and the non-magnetic attraction area 620 may be located at two separated plates or may be located at two separable portions of a plate.

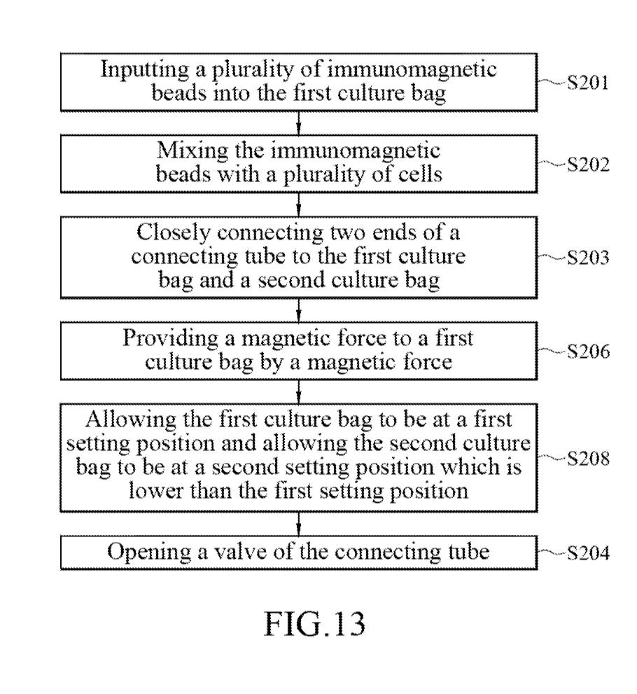

FIG. 13 illustrates a flowchart of a fifth embodiment of a close-system cell isolation method according to the instant disclosure. In some embodiments, the immunomagnetic beads 20 are inputted into the first culture bag 10 (step S201), and then the immunomagnetic beads 20 are mixed with the cells 40 (step S202). Please refer to FIG. 13, before the step S206, two ends of the connecting tube 30 may be closely connected to the first culture bag 10 and the second culture bag 20, respectively, in advance (step S203).

In some embodiments, the valve 311 of the connecting tube 30 may be opened (step S204) after the step S208, so that the first culture bag 10 and the second culture bag 12 are in communication with each other through the tube 300 of the connecting tube 30. Therefore, the valve 311 can be opened to allow the first culture bag 10 being in communication with the second culture bag 12 through the connecting tube 30 after the first culture bag 10 is forced by the magnetic force (namely, after the immunomagnetic beads 20 attracted with the first cells 44 are attached to the magnetic device). Consequently, when the first cells 44 are not attracted by the magnetic force, the first cells 44 would not be mistakenly transmitted to the second culture bag 12.

It is realized that, the order of the steps described above is not a limitation of the instant disclosure, if it is rational, the order of some of the steps may be exchanged, or some of the steps may be performed at the same time. For example, the step S202 may be performed before or after the step S203.

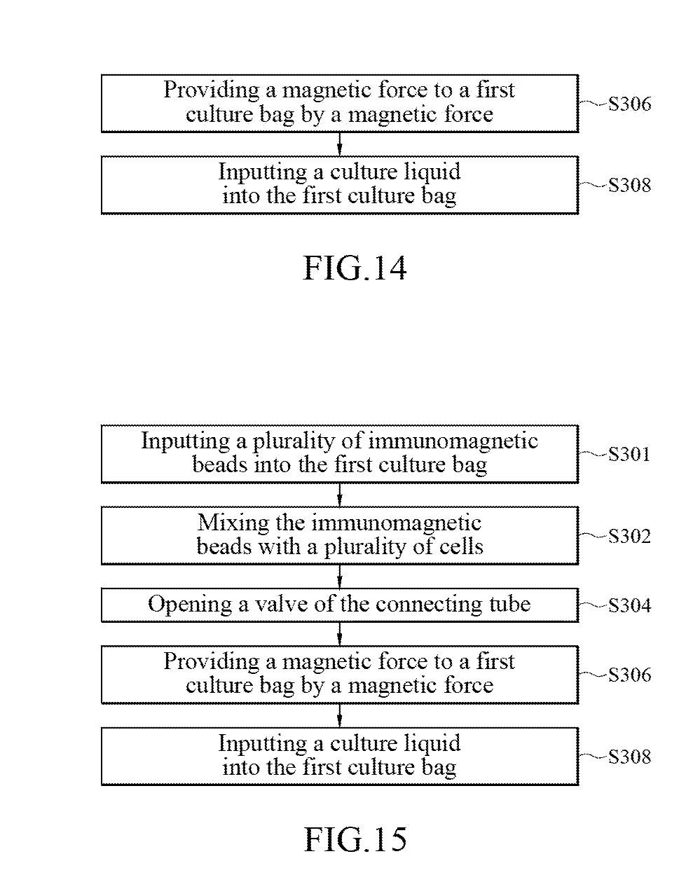

FIG. 14 illustrates a flowchart of a sixth embodiment of a close-system cell isolation method according to the instant disclosure. In some embodiments, as shown in FIG. 14, the close-system cell isolation method comprises providing the magnetic force to the first culture bag 10 by the magnetic device (step S306) and inputting the culture liquid 42 into the first culture bag 10, so that the second cells 46, which are uncaptured by the immunomagnetic beads 20, are moved from the first culture bag 10 to the second culture bag 12 through the connecting tube 30 (step S308). The flowing force produced by the fluid is one of the embodiments of the external force. Therefore, the second cells 46, which are uncaptured by the immunomagnetic beads 20, in the first culture bag 10 are flowed through the connecting tube 30 and further flowed into the second culture bag 12 along with the culture liquid 42; while the first cells 44, which are captured by the immunomagnetic beads 20, are attracted by the magnetic force and retained in the first culture bag 10.

In one embodiment of the step S308, the culture liquid 42 may be inputted into the first culture bag 10 through the first normal close opening 102 or the second normal close opening 103.

FIG. 15 illustrates a flowchart of a seventh embodiment of a close-system cell isolation method according to the instant disclosure. In some embodiments, the immunomagnetic beads 20 may be received in the receiving space 105 of the first culture bag 10 when the first culture bag 10 is purchased. In some embodiments, please refer to FIG. 15, before the step S306, the immunomagnetic beads 20 are inputted into the first culture bag 10 through at least one of the first normal close opening 102 and the second normal close opening 103 of the first culture bag 10 (S301), and then, the immunomagnetic beads 20 and cells 40 are mixed to allow the binders 22 capturing the first cells 44 of the cells 40 (step S302). Next, after the reaction (when the binders 22 are thoroughly combined with the first cells 44), the step S306 and steps after the step S306 are performed.

Please refer to FIG. 15. In some embodiments, the culture bags 10, 12 and the connecting tube 30 are already connected with each other as being purchased. The valve 311 of the connecting tube 30 may be closed to cease the communication between the receiving space 105 of the first culture bag 10 and the receiving space 125 of the second culture bag 12. In the cell isolation procedure, the valve 311 of the connecting tube 30 is opened prior to the step S306, so that the receiving space 105 of the first culture bag 10 and the receiving space 125 of the second culture bag 12 are in communication with each other through the tube 300. Then, step S306 and steps after the step S306 are performed.

In different embodiments, the external force may be oriented from different sources, that is, the external force may be the centrifugation force, the gravity (the difference between the gravitational potential energies), the flowing force of the fluid, or the combination thereof. The external force is applied to allow the second cells 46, which are uncaptured by the immunomagnetic beads 20, to be moved from the first culture bag 10 to the second culture bag 12 or the second culture bag 13 through the connecting tube 30. The magnetic force may be applied to the first culture bag 10 and the connecting tube 30 at the same time to control the moving direction of the second cells 46. It is understood that, aforementioned descriptions illustrate different combinations of embodiments of the instant disclosure, but not a limitation of the instant disclosure. Some of the steps and/or some of the components of the device described above can be exchanged or altered to approach the purpose of the instant disclosure, if it is rational.

As above, a close culture system can be provided, and the cells inside the system can be isolated and purified by the culture bags closely connected with each other without the contamination and provided for further cell culture procedure.

For example, in the culture of cells having multiple states (e.g., the immune cells), the primary cells are inputted to and cultured in the first culture bag 10. After a time duration (for example, several days), the first culture bag 10 will have cells having at least two states (for example, first cells 44 and second cells 46). In the cell isolation procedure, the immunomagnetic beads 20 are utilized to choose the cells having the first state (for example, the first cells 44) so that the first cells 44 are retained in the first culture bag 10, and the cells having the second state (for example, the second cells 46) are separated and inputted into the second culture bag 12. After the separation or isolation, the first cells 44 in the first culture bag 10 and the second cells 46 in the second culture bag 12 may be utilized for further cell culture or for subsequent experiments, respectively.

While the instant disclosure has been described by the way of example and in terms of the preferred embodiments, it is to be understood that the invention need not be limited to the disclosed embodiments. On the contrary, it is intended to cover various modifications and similar arrangements included within the spirit and scope of the appended claims, the scope of which should be accorded the broadest interpretation so as to encompass all such modifications and similar structures.

What is claimed is:

1. A close-system cell isolation device, comprising:
a plurality of culture bags, each of the culture bags comprises:
a bag body, having a receiving space for culturing a plurality of cells;
two normal close openings at the bag body and communicating with the receiving space for inputting or outputting a culture liquid of the cells; and
an normal close connecting port at the bag body and communicating with the receiving space;
a plurality of immunomagnetic beads in the receiving space of a first culture bag of the culture bags, wherein each of the immunomagnetic beads comprises a plurality of binders for capturing a plurality of numbers of the cells; and
a connecting tube, closely connected between the normal close connecting port of the first culture bag and the normal close connecting port of at least one second culture bag of the culture bags;
wherein, when the first culture bag is forced by a magnetic force and an external force, cells uncaptured by the immunomagnetic beads are moved to one of the at least one second culture bag through the connecting tube by the external force.

2. The close-system cell isolation device according to claim 1, wherein the number of the at least one second culture bag is one, the connecting tube is a one-to-one tube, and two ends of the one-to-one tube are closely connected to the normal close connecting port of the first culture bag and the normal close connecting port of the second culture bag, respectively.

3. The close-system cell isolation device according to claim 1, wherein the number of the at least one second culture bag is plural, the connecting tube is a one-to-many tube, a first end of the one-to-many tube is closely connected to the normal close connecting port of the first culture bag, and a plurality of second ends of the one-to-many tube are respectively and closely connected to the normal close connecting ports of the second culture bags.

4. The close-system cell isolation device according to claim 1, wherein the connecting tube has a tube and a valve, the first culture bag and the at least one second culture bag are in communication with each other through the tube, and the valve controls the flow of the tube.

5. The close-system cell isolation device according to claim 1, wherein the two normal close openings and the normal close connecting port of each of the culture bags are at the same side of the culture bag.

6. The close-system cell isolation device according to claim 1, wherein the two normal close openings and the normal close connecting port of each of the culture bags are at opposite sides of the culture bag.

7. The close-system cell isolation device according to claim 1, further comprising:
a magnetic device for providing the magnetic force to the first culture bag; and
a rotating plate for providing the external force to the first culture bag by rotating the first culture bag.

8. The close-system cell isolation device according to claim 1, further comprising:
a magnetic device for providing the magnetic force to the first culture bag; and
an altitude adjusting device having a first setting position and at least one second setting position which is lower than the first setting position, wherein the first culture bag is at the first setting position, and the at least one second culture bag is at the at least one second setting position, so that the external force is formed in the first culture bag.

9. The close-system cell isolation device according to claim 1, further comprising:
a magnetic device for providing the magnetic force to the first culture bag, wherein when one of the two normal close openings of the first culture bag is closed and the normal close connecting port communicates with the connecting tube, the culture liquids are inputted into the other normal close opening of the first culture bag for generating the external force.

10. A close-system cell isolation method, comprising:
providing a magnetic force to a first culture bag by a magnetic device, wherein the first culture bag has a plurality of cells and a plurality of immunomagnetic beads, each of the immunomagnetic beads comprises a plurality of binders for capturing a plurality of numbers of the cells; and
providing a centrifugation force to the first culture bag and a second culture bag communicating with the first culture bag through a connecting tube by the rotation of a rotating plate, so that cells uncaptured by the immunomagnetic beads are moved from the first culture bag to the second culture bag through the connecting tube.

11. The close-system cell isolation method according to claim 10, wherein the connecting tube is a one-to-many tube, a first end of the one-to-many tube is closely connected to the first culture bag, and a plurality of second ends of the one-to-many tube are respectively connected to the second culture bag and another second culture bag.

12. The close-system cell isolation method according to claim 11, further comprising:
providing the magnetic force to the connecting tube by the magnetic device, wherein when the centrifugation force is provided to the first culture bag, a plurality of numbers of the immunomagnetic beads is moved from the first culture bag to the another second culture bag through the connecting tube.

13. A close-system cell isolation method, comprising:
providing a magnetic force to a first culture bag by a magnetic device, wherein the first culture bag has a plurality of cells and a plurality of immunomagnetic beads, each of the immunomagnetic beads comprises a plurality of binders for capturing a plurality of numbers of the cells; and
allowing the first culture bag to be at a first setting position and allowing a second culture bag to be at a second setting position which is lower than the first setting position, so that cells uncaptured by the immunomagnetic beads are moved from the first culture bag to the second culture bag through a connecting tube.

14. The close-system cell isolation method according to claim 13, wherein the connecting tube is a one-to-many tube, a first end of the one-to-many tube is closely connected to the first culture bag, and a plurality of second ends of the one-to-many tube are respectively connected to the second culture bag and another second culture bag.

15. The close-system cell isolation method according to claim 14, further comprising:
providing the magnetic force to the connecting tube by the magnetic device; and
allowing the another second culture bag to be at the second position, wherein when the first culture bag is higher than the another second culture bag, a plurality of numbers of the immunomagnetic beads is moved from the first culture bag to the another second culture bag through the connecting tube.

16. A close-system cell isolation method, comprising:
providing a magnetic force to a first culture bag by a magnetic device, wherein the first culture bag has a plurality of cells and a plurality of immunomagnetic beads, each of the immunomagnetic beads comprises a plurality of binders for capturing a plurality of numbers of the cells; and
inputting a culture liquid into the first culture bag so that cells uncaptured by the immunomagnetic beads are moved from the first culture bag to a second culture bag through a connecting tube.

17. The close-system cell isolation method according to claim 16, wherein the connecting tube is a one-to-many tube, a first end of the one-to-many tube is closely connected to the first culture bag, and a plurality of second ends of the one-to-many tube are respectively connected to the second culture bag and another second culture bag.

18. The close-system cell isolation method according to claim 17, further comprising:
providing the magnetic force to the connecting tube by the magnetic device, wherein when the culture liquid is inputted into the first culture bag, a plurality of numbers of the immunomagnetic beads is moved from the first culture bag to the another second culture bag through the connecting tube.

19. The close-system cell isolation method according to claim 16, further comprising:
opening a valve of the connecting tube to allow a tube of the connecting tube communicating with the first culture bag and the second culture bag.

20. The close-system cell isolation method according to claim 16, wherein the connecting tube is a one-to-one tube, and two ends of the one-to-one tube are closely connected to the first culture bag and the second culture bag, respectively.
